(19) United States
(12) Patent Application Publication
(10) Pub. No.: US 2011/0315939 A1
(43) Pub. Date: Dec. 29, 2011

(54) NEAR INFRARED ABSORBENT DYE AND NEAR INFRARED SHIELDING FILTER

(75) Inventors: Akinori Okayasu, Gunma (JP); Susumu Yamanobe, Gunma (JP); Masaaki Tamura, Gunma (JP); Yoji Yamaguchi, Gunma (JP)

(73) Assignee: Japan Carlit Co., Ltd., Tokyo (JP)

(21) Appl. No.: 13/202,509

(22) PCT Filed: Feb. 18, 2010
(86) PCT No.: PCT/JP10/52422

§ 371 Date: Aug. 19, 2011

(30) Foreign Application Priority Data
Feb. 20, 2009 (JP) 2009-037367

(51) Int. Cl. G02B 5/22; C07C 211/56; C07C 211/55

(52) U.S. Cl. 252/587; 564/434

(57) ABSTRACT

A near infrared absorbent dye is provided, having excellent heat resistance and excellent moisture resistance, as is a near infrared shielding filter using the dye. The near infrared absorbent dye contains an association form of a diimmonium salt compound represented by formula (1):

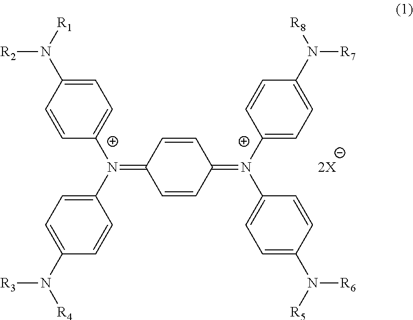

wherein R₁ to R₈ are the same or different and each represents an organic group, and X⁻ represents an anion.

### TECHNICAL FIELD

The present invention relates to a near infrared absorbent dye having an absorption in the near infrared region and a near infrared shielding filter using the dye, and more particularly to a near infrared absorbent dye having excellent infrared absorption effect as well as excellent heat resistance and excellent moisture resistance and a near infrared shielding filter containing the dye.

### BACKGROUND ART

In recent years, there are increasing demands for display having an increased size and a reduced thickness, and plasma display panels (hereinafter, abbreviated to “PDPs”) are generally spreading widely. PDP emits near infrared rays and causes an electronic device using a near infrared remote control to malfunction, and therefore it is necessary to intercept near infrared rays with a filter using a near infrared absorbent dye. Near infrared shielding filters are also widely used in the applications of optical lens, glass for automobile, glass for construction, and the like. The near infrared shielding filters used in these applications are required to effectively absorb rays in the near infrared region while transmitting rays in the visible light region and further have high heat resistance, high moisture resistance, high light resistance, and the like.

As near infrared absorbent dyes absorbing near infrared rays, conventionally, cyanine dyes, polymethine dyes, squarylium dyes, porphyrin dyes, metal dithiol complex dyes, phthalocyanine dyes, diimmonium dyes, inorganic oxide particles, and the like have been used. Among these dyes, diimmonium dyes have high absorptive power for the near infrared rays and high transparency in the visible light region and hence have been widely used (see, for example, Patent document 1). In this patent document, various examples of diimmonium salt near infrared absorbent dyes are shown and, of these, an N,N,N′,N′-tetrakis{p-di(n-butyl)aminophenyl}-p-phenylenediimmonium salt having, for example, bis(hexafluoroantimonate) as an anion component, which is relatively good in heat resistance and moisture resistance, is generally used.

However, this diimmonium salt compound has problems not only in that the heat resistance and moisture resistance are unsatisfactory such that the dye decomposes during the use, causing the near infrared absorptive power to be poor, but also in that an aminium salt formed due to the decomposition of the dye exhibits an absorption in the visible light region and thus the visible light transmission is lowered to cause yellowing, so that the color tone deteriorates.

Further, Patent document 2 discloses an infrared absorbing film containing an organic solvent-soluble diimmonium dye in a state in which fine particles of the dye are dispersed in a resin.

With respect to the diimmonium dye disclosed in this patent document, however, especially in a resin having a low glass transition temperature, such as an adhesive resin, the molecular interaction of the organic solvent-soluble dye is so weak that the dye is likely to suffer marked deterioration, and hence the dye is poor in respect of practical use. Further, the dye disclosed in the document has so poor dispersion stability that the crystals of the dye easily become coarse, and therefore the dye disadvantageously exhibits an absorption band having a large half band width and a low absorption coefficient at the absorption maximum. For this reason, when this dye is used in a near infrared shielding filter, there occur problems not only in that a satisfactory near infrared absorption effect cannot be obtained, but also in that the coarse crystals of the dye scatter rays of light to cause the filter to be opaque.

### RELATED ART DOCUMENTS

### Patent Documents

Patent document 1: JP-A-10-180922
Patent document 2: Japanese Patent No. 3987240

### SUMMARY OF THE INVENTION

### Problems to be Solved by the Invention

Accordingly, there is a need for the development of a near infrared absorbent dye having further excellent heat resistance and excellent moisture resistance, and the present invention has an object to provide a near infrared absorbent dye having such excellent properties and a near infrared shielding filter using the dye.

### Means for Solving the Problems

The present inventors have conducted extensive and intensive studies with a view toward solving the above-mentioned problems. As a result, they have found that an association form of diimmonium exhibits high near infrared absorptive power as well as excellent heat resistance and excellent moisture resistance, and the present invention has been completed.

Specifically, the present invention is directed to a near infrared absorbent dye containing an association form of a diimmonium salt compound represented by the following general formula (1):

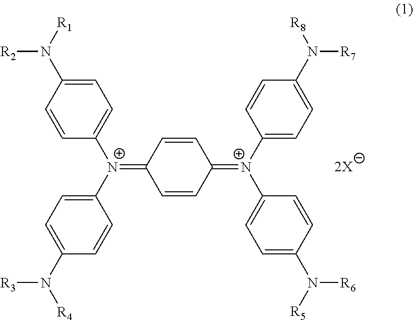

wherein R₁ to R₈ are the same or different and each represents an organic group, and X represents an anion.

The invention is the near infrared absorbent dye wherein X in the general formula (1) above is a hexafluorophosphate ion.

The invention is the near infrared absorbent dye wherein at least one of R₁ to R₈ in the general formula (1) above is a cycloalkyl-alkyl group represented by the following general formula (2):

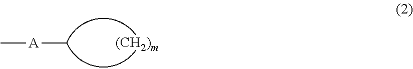

wherein A represents a linear or branched alkyl group having 1 to 10 carbon atoms, and m represents an integer of 3 to 12.

The invention is the near infrared absorbent dye wherein at least one of R₁ to R₈ in the general formula (1) above is a monohalogenoalkyl group represented by the following general formula (3):

—CₙH₂ₙ—CH₂Y (3)

wherein n represents an integer of 1 to 9, and Y represents a halogen atom.

The invention is the near infrared absorbent dye wherein at least one of R₁ to R₈ in the general formula (1) above is an iso-butyl group.

Further, the invention is directed to a near infrared absorbing composition containing the diimmonium salt compound represented by the general formula (1) above, which is in an association state dispersed in an organic solvent.

Furthermore, the invention is directed to a near infrared shielding filter containing the above-mentioned near infrared absorbent dye.

### Effects of the Invention

The near infrared absorbent dye of the present invention has a high absorption coefficient at the absorption maximum and excellent near infrared absorptive power as well as excellent heat resistance and excellent moisture resistance. By using this dye, it becomes possible to provide a near infrared shielding filter which is advantageous not only in that the filter is unlikely to scatter rays of light and hence has excellent transparency, but also in that the filter can maintain high near infrared absorptive power over a longer term.

### BRIEF DESCRIPTION OF THE DRAWINGS

FIG. 1 shows absorption spectra measured in Test Example 1 with respect to the dispersions or solutions of the diimmonium salt compounds obtained in Production Examples 1 to 3 and Comparative Production Examples 1 and 2 at a concentration of 100 mg/L.

FIG. 2 shows molar absorption coefficients measured in Test Example 1 with respect to the dispersions or solutions of the diimmonium salt compound obtained in Production Example 1 at the respective concentrations.

FIG. 3 shows molar absorption coefficients measured in Test Example 1 with respect to the dispersions of the diimmonium salt compound obtained in Production Example 2 at the respective concentrations.

FIG. 4 shows a molar absorption coefficient measured in Test Example 1 with respect to the solution of the diimmonium salt compound obtained in Production Example 2 at a concentration of 10 mg/L, which solution is obtained by dilution with methylene chloride.

FIG. 5 shows molar absorption coefficients measured in Test Example 1 with respect to the dispersions or solutions of the diimmonium salt compound obtained in Production Example 3 at the respective concentrations.

FIG. 6 shows a molar absorption coefficient measured in Test Example 1 with respect to the dispersion of the diimmonium salt compound obtained in Comparative Production Example 1 at a concentration of 5 mg/L.

FIG. 7 shows a molar absorption coefficient measured in Test Example 1 with respect to the solution of the diimmonium salt compound obtained in Comparative Production Example 1 at a concentration of 10 mg/L, which solution is obtained by dilution with methylene chloride.

FIG. 8 shows a molar absorption coefficient measured in Test Example 1 with respect to the solution of the diimmonium salt compound obtained in Comparative Production Example 2 at a concentration of 100 mg/L.

### MODE FOR CARRYING OUT THE INVENTION

The near infrared absorbent dye of the present invention contains an association form of a diimmonium salt compound represented by the general formula (1) below {hereinafter, frequently referred to as “diimmonium salt compound (1)”}. In the present specification, the term “near infrared rays” means rays of light having a wavelength in the range of from 750 to 2,000 nm.

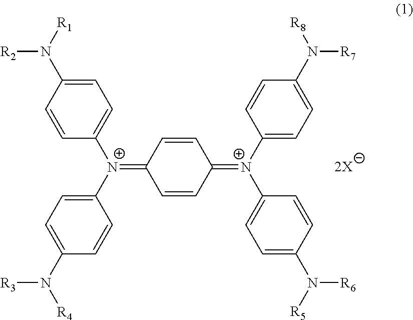

wherein R₁ to R₈ are the same or different and each represents an organic group, and X represents an anion.

In the general formula (1) above, the organic groups R₁ to R₈ may be the same or different, and they are not particularly limited as long as an association form of the compound can be made. Preferred examples of the organic groups include a linear or branched C₁-C₁₀ alkyl group optionally substituted with a halogen atom, a C₃-C₁₂ cycloalkyl group, and a C₃-C₁₂ cycloalkyl-C₁-C₁₀ alkyl group in which the cycloalkyl ring is optionally substituted. At least one of R₁ to R₈ may be the above organic group, but it is preferred that all R₁ to R₈ be the same and one of the above organic groups because the resultant cation structure becomes symmetric, facilitating the arrangement.

Examples of the linear or branched C₁-C₁₀ alkyl groups include a methyl group, an ethyl group, a n-propyl group, an iso-propyl group, a n-butyl group, an iso-butyl group, a sec-butyl group, a tert-butyl group, a n-amyl group, an iso-amyl group, a 1-methylbutyl group, a 2-methylbutyl group, a 1-ethylbutyl group, a 2-ethylbutyl group, a 2-dimethylpropyl group, a 1,1-dimethylpropyl group, a neopentyl group, and a n-hexyl group. Of these, preferred are branched C₃-C₆ alkyl groups, such as an iso-propyl group, an iso-butyl group, and an iso-amyl group, from the viewpoint of obtaining the arrangement of molecules required for the formation of an association form of the compound, and an iso-butyl group is especially preferred.

Examples of the C₃-C₁₂ cycloalkyl groups include a cyclopentyl group and a cyclohexyl group.

In the C₃-C₁₂ cycloalkyl-C₁-C₁₀ alkyl group, the cycloalkyl ring may be substituted or unsubstituted, and examples of substituents for the substitution include an alkyl group, a hydroxyl group, a sulfonic acid group, an alkylsulfonic acid group, a nitro group, an amino group, an alkoxy group, a halogenoalkyl group, and a halogen atom. The cycloalkyl ring is preferably unsubstituted, and a cycloalkyl-alkyl group represented by the general formula (2) below is preferred because the arrangement of molecules required for the formation of an association form of the compound can be easily obtained.

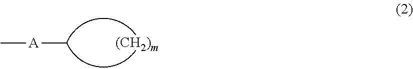

wherein A represents a linear or branched alkyl group having 1 to 10 carbon atoms, and m represents an integer of 3 to 12.

In the general formula (2) above, A preferably has 1 to 4 carbon atoms, and m is preferably 5 to 8, especially preferably 5 to 6. When the values fall inside these ranges, an increased molecular interaction needed for the association is obtained. Specifically, examples include a cyclopentylmethyl group, a 2-cyclopentylethyl group, a 2-cyclopentylpropyl group, a 3-cyclopentylpropyl group, a 4-cyclopentylbutyl group, a 2-cyclohexylmethyl group, a 2-cyclohexylethyl group, a 3-cyclohexylpropyl group, and a 4-cyclohexylbutyl group. Of these, preferred are a cyclopentylmethyl group, a cyclohexylmethyl group, a 2-cyclohexylethyl group, a 2-cyclohexylpropyl group, a 3-cyclohexylpropyl group, and a 4-cyclohexylbutyl group, and more preferred are a cyclopentylmethyl group and a cyclohexylmethyl group, and a cyclohexylmethyl group is especially preferred.

Examples of the linear or branched C₁-C₁₀ alkyl groups substituted with a halogen atom include halogenoalkyl groups, such as a 2-halogenoethyl group, a 2,2-dihalogenoethyl group, a 2,2,2-trihalogenoethyl group, a 3-halogenopropyl group, a 3,3-dihalogenopropyl group, a 3,3,3-trihalogenopropyl group, a 4-halogenobutyl group, a 4,4-dihalogenobutyl group, a 4,4,4-trihalogenobutyl group, a 5-halogenopentyl group, a 5,5-dihalogenopentyl group, and a 5,5,5-trifluoropentyl group. Of these, preferred is a monohalogenoalkyl group represented by the following general formula (3):

—CₙH₂ₙ—CH₂Y (3)

wherein n represents an integer of 1 to 9, and Y represents a halogen atom.

In the general formula (3), n is preferably 1 to 4, and Y is especially preferably a fluorine atom. When the value falls inside this range, an increased molecular interaction needed for the association is obtained. Specifically, examples include monofluoroalkyl groups, such as a 2-fluoroethyl group, a 3-fluoropropyl group, a 4-fluorobutyl group, and a 5-fluoropentyl group. More preferred are a 3-fluoropropyl group, a 4-fluorobutyl group, and a 5-fluoropentyl group, and a 3-fluoropropyl group is especially preferred.

In the above-mentioned diimmonium salt compound (1), a diimmonium salt compound of the general formula (4) below in which all R₁ to R₈ are cyclohexylmethyl groups and a diimmonium salt compound of the general formula (5) below in which all R₁ to R₈ are 3-fluoropropyl groups are novel compounds. These diimmonium salt compounds are in an association form, particularly excellent in heat resistance and moisture resistance and have high near infrared absorptive power, and therefore they are preferably used.

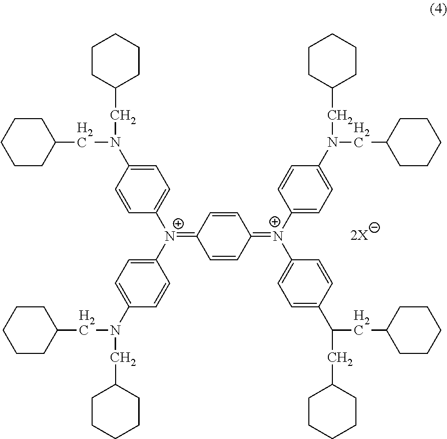

wherein X⁻ represents an anion.

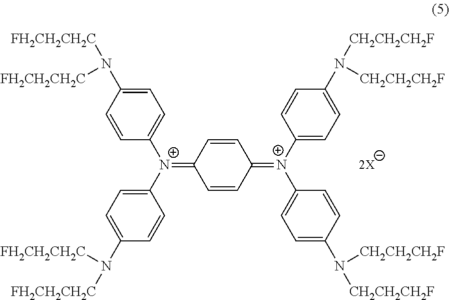

wherein X⁻ represents an anion.

On the other hand, X⁻ in the general formula (1) is an anion required for neutralization of the charge of the diimmonium cation, and an organic acid anion, an inorganic anion, or the like can be used. An inorganic anion is preferred because it lowers the solubility of the diimmonium salt to facilitate the formation of an association form of the diimmonium salt. Specific examples of inorganic anions include halogen ions, such as a fluorine ion, a chlorine ion, a bromine ion, and an iodine ion, a perchlorate ion, a periodate ion, a tetrafluoroborate ion, a hexafluorophosphate ion, and a hexafluoroantimonate ion. A hexafluorophosphate ion is especially preferred because the arrangement of molecules required for the formation of an association form of the compound can be easily obtained.

The diimmonium salt compound (1) used in the present invention can be produced by the following method.

Specifically, to an amino compound obtained by an Ullmann reaction and a reduction reaction, which is represented by the formula (6) below, in a polar solvent, such as N-methyl-2-pyrrolidone (hereinafter, abbreviated to “NMP”) or dimethylformamide (hereinafter, abbreviated to “DMF”), are added an iodide or iodides corresponding to R₁ to R₈ and an alkylmetal carbonate as a deiodination agent, and the resultant mixture is subjected to reaction at 30 to 150° C., preferably at 70 to 120° C. to obtain an alkyl-substituted compound represented by the formula (7) below. For example, when all R₁ to R₈ are cyclohexylmethyl groups, an iodocyclohexylalkane as a corresponding iodide is reacted with the amino compound, and when all R₁ to R₈ are 3-fluoropropyl groups, an iodofluoroalkane is reacted with the amino compound. On the other hand, when R₁ to R₈ are two or more different organic groups, iodides in moles corresponding to the respective numbers of the organic groups are successively reacted with the amino compound in the same manner as mentioned above or added at the same time and reacted with the amino compound to obtain the compound of the formula (7). For example, when R₁ to R₈ are a cyclohexylmethyl group(s) and another or other organic group or groups, an iodocyclohexylalkane in a mole corresponding to the number of the substituent(s) is added and reacted with the amino compound, and after the reaction, an iodide or iodides (e.g., an iodofluoroalkane, an iodoalkane, an alkoxyiodide, an iodobenzene, or a phenyl-1-iodoalkane, such as an iodobenzyl or an iodophenethyl) in the corresponding moles is or are successively added and reacted with the amino compound, or these different iodides are added at the same time and reacted with the amino compound to obtain the compound of the formula (7).

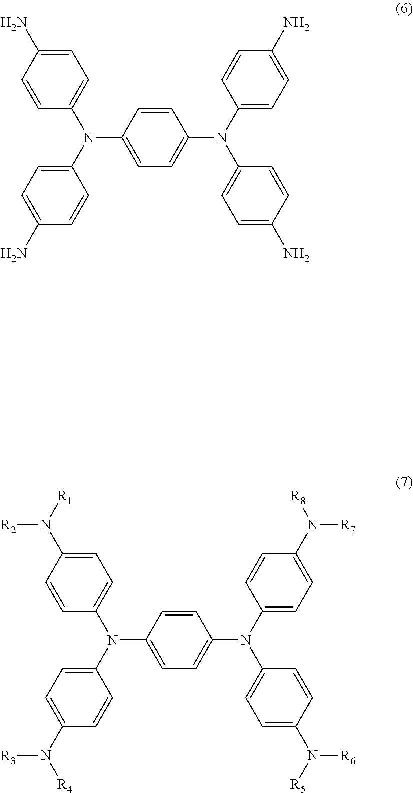

wherein R₁ to R₈ have the same meaning as mentioned above.

Then, the alkyl-substituted compound represented by the formula (7) above and a silver salt of the corresponding anion X⁻ are reacted with each other in an organic solvent, such as NMP, DMF, or acetonitrile, at a temperature of 30 to 150° C., preferably 40 to 80° C. The deposited silver is filtered off, and then a solvent, such as water, ethyl acetate, or hexane, is added to the filtrate and the resultant precipitate is collected by filtration, thus obtaining the diimmonium salt compound (1).

The near infrared absorbent dye of the present invention contains an association form of the thus obtained diimmonium salt compound (1), and is characterized by an absorption in the wavelength region of 750 to 1,300 nm and a maximum absorption wavelength in 1,110 to 1,250 nm. Further, the near infrared absorbent dye of the invention has the maximum absorption wavelength which has shifted 15 to 200 nm to the longer wavelength side from the maximum absorption wavelength of the diimmonium salt compound in a dissolved state.

Specifically, it has been known that a dye compound in an association state (in a state of being dispersed as an association form) forms a so-called association band and exhibits an absorption spectrum different from that of the dye compound in a dissolved state {for example, Photographic Science and Engineering, Vol. 18, No. 323-335 (1974)}, and, generally, the absorption band of the dye compound in an association state shifts to the longer wavelength side from the absorption band of the dye compound in a dissolved state. Whereas a diimmonium salt compound in a dissolved state generally exhibits a maximum absorption wavelength in 1,050 to 1,095 nm, the near infrared absorbent dye of the present invention has an association form of the diimmonium salt compound and therefore exhibits an absorption maximum wavelength in 1,110 to 1,250 nm, as a result of the shifting 15 to 200 nm to the longer wavelength side from the above-mentioned wavelength of the diimmonium salt compound in a dissolved state. When the amount of change in the shifting is too large, the near infrared absorption at about 900 to 1,100 nm may be unsatisfactory, and therefore the change is preferably 15 to 100 nm.

The absorption wavelength region and maximum absorption wavelength of the near infrared absorbent dye of the present invention are determined from an absorption spectrum of the diimmonium salt compound as measured in a state in which the diimmonium salt compound is suspended in the form of 0.001 to 10 μm (10⁻⁹ m to 10⁻⁵ m) particles in a dispersing medium at a concentration of at least 50 mg/L (hereinafter, this state is frequently referred to as “dispersed state”). The particle size is measured by means of Microtrac Particle Size Analyzer. More specifically, the absorption wavelength region and maximum absorption wavelength are determined from an absorption spectrum as measured by means of a spectrophotometer with respect to the diimmonium salt compound dispersion obtained by a method in which 0.5 part of the diimmonium salt compound, 9.5 parts of toluene, and 70 parts of zirconia beads having a particle size of 0.3 mm are placed in a 50-ml glass vessel and subjected to shaking by means of a paint shaker for 2 hours, and then the zirconia beads are filtered off and the resultant filtrate is diluted with toluene so that the concentration of the diimmonium salt compound in the resultant dispersion becomes 100 mg/L. On the other hand, the maximum absorption wavelength of the dye in a dissolved state is determined from an absorption spectrum as measured by means of a spectrophotometer with respect to the solution obtained by diluting the above-prepared diimmonium salt compound dispersion with toluene at a concentration such that the diimmonium salt compound is in a dissolved state. In the case where the diimmonium salt compound does not become in a dissolved state even when the dispersion is diluted with toluene at about 5 mg/L, the dispersion is diluted with methylene chloride instead of toluene, and with respect to the resultant solution, an absorption spectrum can be similarly measured.

Further, the diimmonium salt compound may be in the above-mentioned dispersed state not as an association form but as a crystal. The diimmonium salt compound in an association state exhibits a sharp absorption band having a smaller half band width (width of the wavelength region exhibiting half of the absorbance at the absorption maximum) than that of the compound in a crystal dispersed state. In the diimmonium salt compound in a crystal dispersed state, the amount of change of the maximum absorption wavelength from that of the compound in a dissolved state is large, and the maximum absorption wavelength shifts to the wavelength longer than 1,250 nm. Also, with respect to the molar absorption coefficient at the maximum absorption wavelength, the diimmonium salt compound in an association state has a value of 70,000 mol⁻¹ L cm⁻¹ or more (wherein L means the length of a cell), whereas the compound in a crystal dispersed state has a value as small as less than 40,000 mol⁻¹ L cm⁻¹ and thus has markedly poor near infrared absorptive power as compared with the compound in an association state.

An absorption spectrum of the diimmonium salt compound as measured in a dispersed state and that measured in a dissolved state are compared to obtain respective maximum absorption wavelengths and an amount of change between them, thus making it possible to determine whether the diimmonium salt compound is in an association state or in a dissolved state. On the other hand, whether the diimmonium salt compound is in an association state or in a crystal dispersed state may be distinguished by using the maximum absorption wavelength of an absorption spectrum of the diimmonium salt compound as measured in a dispersed state and the molar absorption coefficient at that wavelength obtained for comparison.

The near infrared absorbent dye of the present invention can be obtained in the form of a solid fine particle dispersion having an association form of the above-obtained diimmonium salt compound (1) formed using a known dispersion mixer. Examples of dispersion mixers include a ball mill, a vibration ball mill, a planetary ball mill, a sand mill, a colloid mill, a jet mill, and a roller mill, and the dispersion mixer described in JP-A-52-92716 or International Publication No. 88/074794 pamphlet can also be used. Of these, preferred is a vertical or horizontal medium dispersion mixer. Although the diimmonium salt compound (1) may be dispersed without using a dispersing medium, it is preferred that the compound be dispersed in the presence of a dispersing medium. As a dispersing medium, water or an organic solvent can be used, and an organic solvent is preferred because of its ease of mixing with a coating resin, and especially preferred is a solvent having an affinity with a coating resin, such as toluene or ethyl acetate. Further, a surfactant may be used, and a conventionally known anionic surfactant, anionic polymer, nonionic surfactant, or cationic surfactant can be used. Thus, a near infrared absorbing composition containing the diimmonium salt compound (1) in an association state in a dispersing medium is obtained.

In the near infrared absorbing composition thus obtained, the whole of the diimmonium salt compound (1) may exist in an association form, or only part of the compound may form in an association form and the rest may remain in a dissolved state or in a crystal dispersed state, depending on the concentration of the diimmonium salt compound in the composition or the like. In any case, the near infrared absorbing composition of the present invention includes the near infrared absorbing composition having a maximum absorption wavelength in the range of from 1,110 to 1,250 nm and a molar absorption coefficient of 70,000 mol⁻¹ L cm⁻¹ or more at the maximum absorption wavelength.

The near infrared shielding filter of the present invention can be prepared in the form of a film or a panel from a combination of the above-mentioned near infrared absorbing composition and an appropriate resin by a known method for preparation, such as a casting method or a melt extrusion method. The casting method is a method in which the near infrared absorbing composition is dispersed in a resin and a solvent, and then the resultant dispersion is applied onto a support, such as a transparent film of polyester, polycarbonate or the like, a panel, or a glass substrate, and dried to form a film. Examples of resins used in the casting method include an acrylic resin, a polyester resin, a polycarbonate resin, an urethane resin, a cellulose resin, a polyisocyanate resin, a polyallylate resin, and an epoxy resin. With respect to the solvent, there is no particular limitation, and an organic solvent, such as methyl ethyl ketone, methyl isobutyl ketone, toluene, xylene, tetrahydrofuran, or 1,4-dioxane, or a mixed solvent thereof can be used. The melt extrusion method is a method in which the near infrared absorbing composition and a resin are melted and kneaded together and then shaped into a panel form by extrusion. The resin used in the melt extrusion method is similar to that in the casting method. Alternatively, the near infrared shielding filter of the present invention can be prepared by directly dispersing the diimmonium salt compound (1) into the above-mentioned resin or solvent using the above-mentioned dispersion mixer to form into a film or to shape by the casting method, the melt extrusion method, or the like, without a process of preparation of the near infrared absorbing composition.

In the preparation of the near infrared shielding filter of the present invention, it is possible to use only the near infrared absorbent dye(s) of the invention, but in the case that the near infrared shielding performance at a wavelength of about 850 nm is slightly unsatisfactory, a known dye, such as a phthalocyanine dye or a dithiol metal complex, may be further added. A benzophenone, benzotriazole or the like ultraviolet absorbing dye may be further added to improve the light resistance. If necessary, a known dye having an absorption in the visible light region may be added to control the color tone of the filter.

The near infrared transmittance of the near infrared shielding filter of the present invention can be controlled by changing the amount of the near infrared absorbent dye of the invention added to the above-mentioned resin. It is preferred that the near infrared absorbent dye of the invention be mixed in an amount in the range of from 0.01 to 30 parts by mass, relative to 100 parts by mass of the resin. When the amount of the dye is less than 0.01 part by mass, the near infrared shielding performance may become unsatisfactory, and when the amount is more than 30 parts by mass, the visible light transmission may be lowered.

The near infrared shielding filter of the present invention can be used in various applications which need to intercept near infrared rays. Specifically, the filter can be used as, for example, a near infrared shielding filter for PDP, a near infrared shielding filter for automobile glass or construction glass, or the like, and is especially preferably used as a near infrared shielding filter for PDP.

Conventionally, when a near infrared absorbent dye containing a diimmonium salt compound is used for a near infrared shielding filter for PDP or the like, the substituents in the diimmonium salt compound are often appropriately selected so that the compound in a dissolved state is used in the dye. Many of such near infrared absorbent dyes, however, have poor durability, which makes it difficult to put the dye into practical use. There is an instance where the diimmonium salt compound in a crystal dispersed state is used in the dye, but such a dye has so poor dispersion stability that the crystals of the compound easily become coarse, and therefore the dye exhibits an absorption band having a large half band width and a low absorption coefficient at the absorption maximum. For this reason, when this dye is used in a near infrared shielding filter, a satisfactory near infrared absorption effect cannot be obtained, and further the coarse crystals of the compound scatter rays of light to cause the filter to be opaque.

In contrast, the near infrared absorbent dye of the present invention consists of an association form of the compound, and hence forms a so-called association band exhibiting a sharp absorption band having a small half band width of the absorption band, and has excellent near infrared absorptive power such that the absorption coefficient at the absorption maximum is high. Further, the near infrared absorbent dye of the invention is considered to have an aggregate consisting of several molecules to several tens of molecules per aggregate, and when used for a near infrared shielding filter, the near infrared shielding filter obtained is unlikely to scatter rays of light and has excellent transparency. Moreover, in terms of an aminium salt compound which is formed by decomposition of a diimmonium salt compound and has an absorption in the visible light region (around 480 nm) leading to yellowing, it is presumed that an association form of the diimmonium salt compound prevents an aminium salt compound from forming because such an aggregation of molecules is stabilized by the molecular interaction as compared with the compound in a monomolecular dispersed state, and thus the compound achieves excellent heat resistance and excellent moisture resistance as well as excellent light resistance.

### EXAMPLES

Hereinbelow, the present invention will be described in more detail with reference to the following Examples, which should not be construed as limiting the scope of the invention. In the Examples, the “part(s)” indicates “part(s) by mass”.

### Production Example 1

### Production of N,N,N′,N′-tetrakis{p-di(cyclohexylmethyl)aminophenyl}-p-phenylenediimmonium hexafluorophosphate

To 100 parts of DMF were added 10 parts of N,N,N′,N′-tetrakis(p-aminophenyl)-p-phenylenediamine, 63 parts of cyclohexylmethyl iodide, and 30 parts of potassium carbonate, and the resultant mixture was subjected to reaction at 120° C. for 10 hours. The reaction mixture was poured into 500 parts of water, and the resultant precipitate was collected by filtration and washed with 500 parts of methyl alcohol, and then dried at 100° C. to obtain 24.1 parts of N,N,N′,N′-tetrakis{p-di(cyclohexylmethyl)aminophenyl}-p-phenylenediamine. In an analysis of the obtained compound by infrared absorption spectroscopy, the absorption ascribed to the NH stretching vibration of the amino group derived from the starting material has disappeared, and the result has confirmed that all the substituents are replaced by cyclohexylmethyl groups.

To 24.1 parts of the obtained N,N,N′,N′-tetrakis{p-di(cyclohexylmethyl)aminophenyl}-p-phenylenediamine were added 200 parts of DMF and 7.9 parts of silver hexafluorophosphate, and the resultant mixture was subjected to reaction at 60° C. for 3 hours, and the resultant silver was filtered off. Then, 200 parts of water was added to the filtrate, and the resultant precipitate was collected by filtration and dried to obtain 27.0 parts of N,N,N′,N′-tetrakis{p-di(cyclohexylmethyl)aminophenyl}-p-phenylenediimmonium hexafluorophosphate.

### Production Example 2

### Production of N,N,N′,N′-tetrakis{p-di(3-fluoropropyl)aminophenyl}-p-phenylenediimmonium hexafluorophosphate

18 Parts of N,N,N′,N′-tetrakis{p-di(3-fluoropropyl)aminophenyl}-p-phenylenediimmonium hexafluorophosphate was obtained in substantially the same manner as in Production Example 1 except that, instead of 63 parts of cyclohexylmethyl iodide, the corresponding number of moles of 1-iodo-3-fluoropropane was used. Further, with respect to the N,N,N′,N′-tetrakis{p-di(3-fluoropropyl)aminophenyl}-p-phenylenediamine obtained in the same manner as in Production Example 1, in an analysis by infrared absorption spectroscopy, the absorption ascribed to the NH stretching vibration of the amino group derived from the starting material has disappeared, and the result has confirmed that all the substituents are replaced by 3-fluoropropyl groups.

### Production Example 3

### Production of N,N,N′,N′-tetrakis{p-di(iso-butyl)aminophenyl}-p-phenylenediimmonium hexafluorophosphate

18 Parts of N,N,N′,N′-tetrakis{p-di(iso-butyl)aminophenyl}-p-phenylenediimmonium hexafluorophosphate was obtained in substantially the same manner as in Production Example 1 except that, instead of 63 parts of cyclohexylmethyl iodide, the corresponding number of moles of isobutyl iodide was used. Further, with respect to the N,N,N′,N′-tetrakis{p-di(iso-butyl)aminophenyl}-p-phenylenediamine obtained in the same manner as in Production Example 1, in an analysis by infrared absorption spectroscopy, the absorption ascribed to the NH stretching vibration of the amino group derived from the starting material has disappeared, and the result has confirmed that all the substituents are replaced by iso-butyl groups.

### Comparative Production Example 1

### Production of N,N,N′,N′-tetrakis{p-di(n-propyl)aminophenyl}-p-phenylenediimmonium hexafluoroantimonate

24.1 Parts of N,N,N′,N′-tetrakis{p-di(n-propyl)aminophenyl}-p-phenylenediamine was obtained in substantially the same manner as in Production Example 1 except that, instead of 63 parts of cyclohexylmethyl iodide, the corresponding number of moles of 1-iodopropane was used.

To 24.1 parts of the obtained N,N,N′,N′-tetrakis{p-di(n-propyl)aminophenyl}-p-phenylenediamine were added 200 parts of DMF and 12.9 parts of silver hexafluoroantimonate, and the resultant mixture was subjected to reaction at 60° C. for 3 hours, and the resultant silver was filtered off. Then, 200 parts of water was added to the filtrate, and the resultant precipitate was collected by filtration and dried to obtain 28.0 parts of N,N,N′,N′-tetrakis{p-di(n-propyl)aminophenyl}-p-phenylenediimmonium hexafluoroantimonate.

### Comparative Production Example 2

### Production of N,N,N′,N′-tetrakis{p-di(n-propyl)aminophenyl}-p-phenylenediimmonium tetrafluoroborate

To the N,N,N′,N′-tetrakis{p-di(n-propyl)aminophenyl}-p-phenylenediamine obtained in the same manner as in Comparative Production Example 1 were added 250 parts of acetone and 14.5 parts of silver tetrafluoroborate, and the resultant mixture was subjected to reaction at 60° C. for 3 hours, and the resultant silver was filtered off. Then, 200 parts of water was added to the filtrate, and the resultant precipitate was collected by filtration and dried to obtain 29.9 parts of a near infrared absorbent dye of N,N,N′,N′-tetrakis{p-di(n-propyl)aminophenyl}-p-phenylenediimmonium tetrafluoroborate.

### Test Example 1

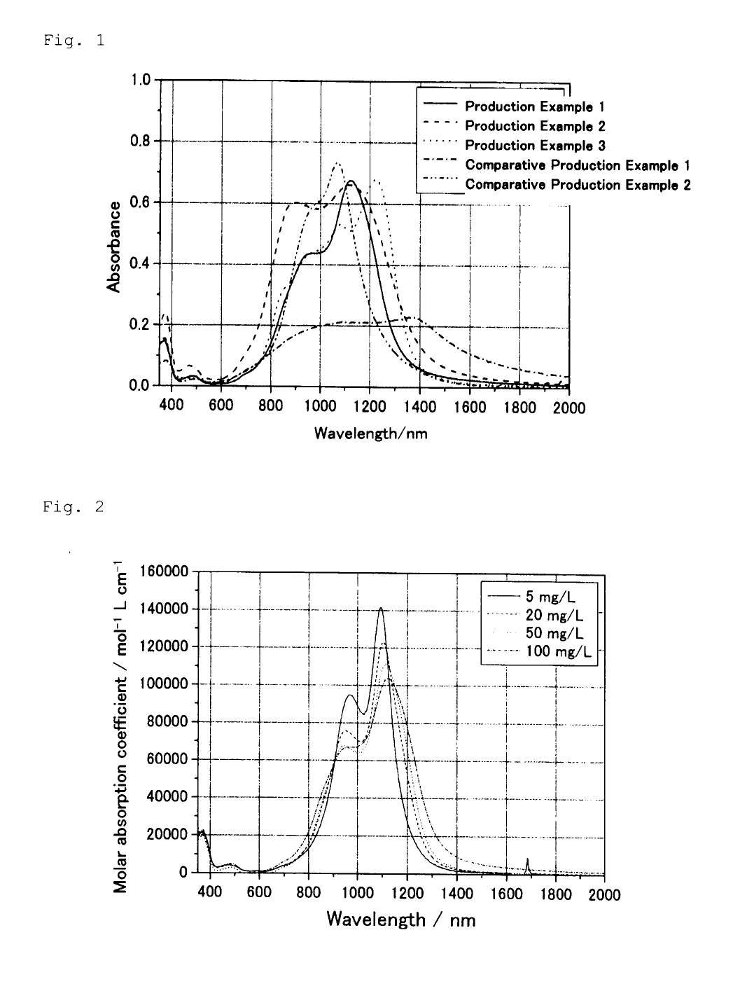

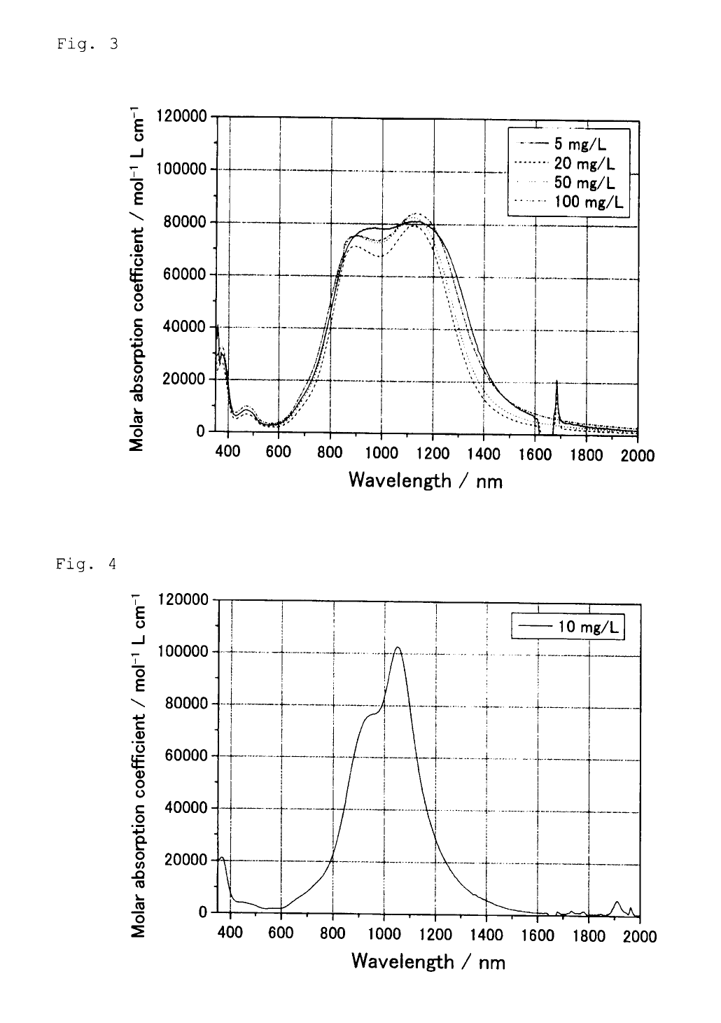

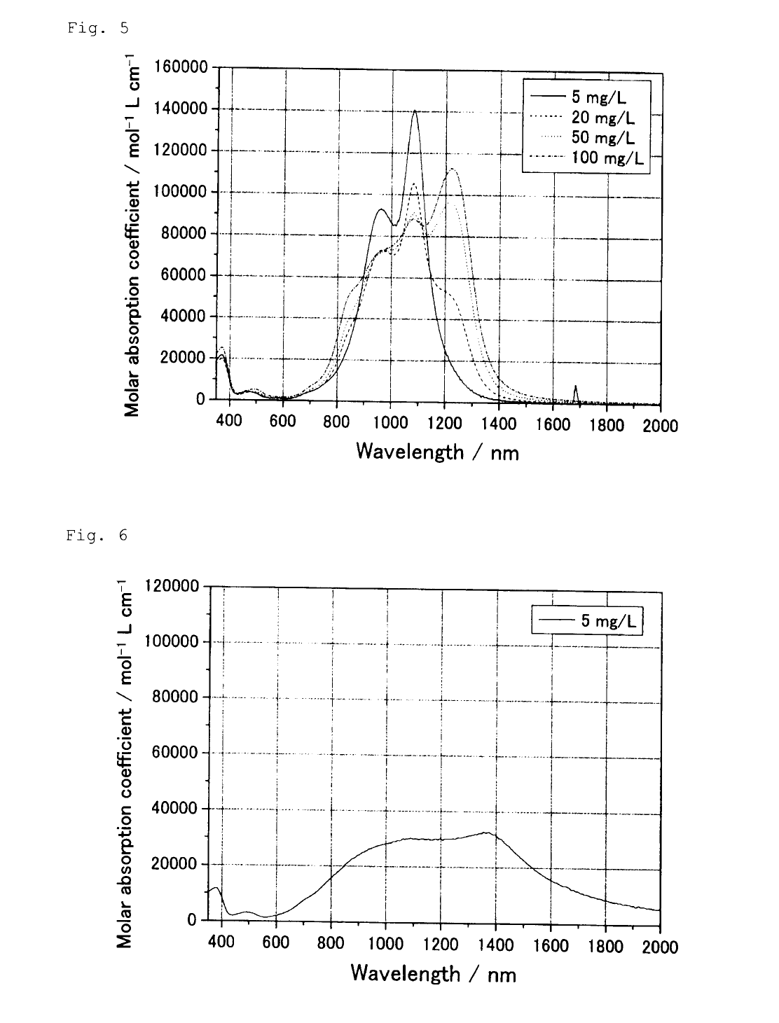

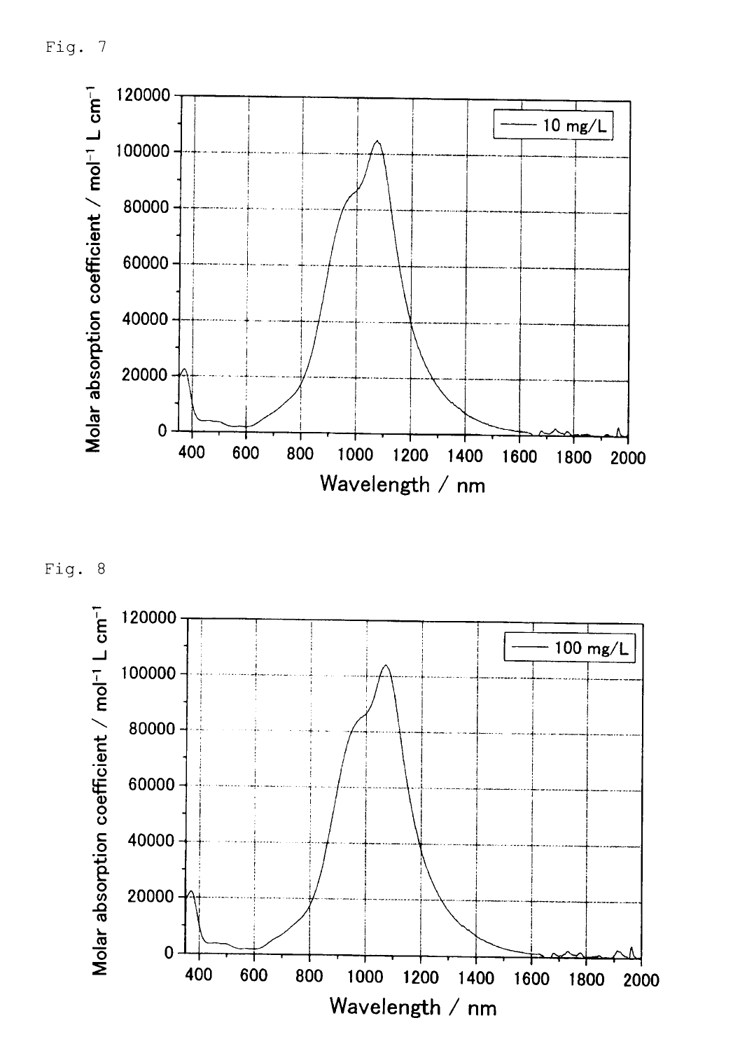

0.5 Part of the N,N,N′,N′-tetrakis{p-di(cyclohexylmethyl)aminophenyl}-p-phenylenediimmonium hexafluorophosphate obtained in Production Example 1, 9.5 parts of toluene, and 70 parts of zirconia beads having a particle size of 0.3 mm were placed in a 50-ml glass vessel and subjected to shaking by means of a paint shaker for 2 hours, and then the zirconia beads were filtered off to prepare a diimmonium salt compound dispersion. The obtained dispersion was diluted with toluene so that the concentration became 5, 20, 50, or 100 mg/L, and an absorbance of each of the diluted dispersions was measured by means of spectrophotometer U-4100 (manufactured by Hitachi High-Technologies Corporation). With respect to each of the diimmonium salt compounds obtained in Production Examples 2 and 3 and Comparative Production Examples 1 and 2, an absorbance was measured similarly. The absorbances of the respective diimmonium salt compounds at a diimmonium salt compound concentration of 100 mg/L are shown in FIG. 1. Since each of the diimmonium salt compounds in Production Example 2 and Comparative Production Example 1 did not become in a dissolved state even when diluted to 5 mg/L and thus was almost insoluble in toluene, a solution of each compound was prepared using methylene chloride as a diluent solvent so that the diimmonium salt compound concentration became 10 mg/L. The molar absorption coefficients of the dispersions or solutions of the respective diimmonium salt compounds at the respective concentrations are shown in FIGS. 2 to 8. Further, the maximum absorption wavelengths of the respective diimmonium salt compounds in a dissolved state and in an association state and the amounts of change in the shifting of the wavelength to the longer wavelength side, and the molar absorption coefficients at the maximum absorption wavelength and half band widths of the respective compounds in a dispersed state are shown in Table 1.

TABLE 1

| | Maximum absorption | | | | |
|---|---|---|---|---|---|
| | wavelength (nm) | | | Molar absorption | Half band |
| | Dissolved | Association | Amount of change | coefficient | width |
| | state | state | (nm) | (mol⁻¹ L cm⁻¹) | (nm) |
| Production Example 1 | 1,094 | 1,119 | 25 | 103,634 | 361 |
| Production Example 2 | 1,050 | 1,120 | 70 | 83,775 | 567 |
| Production Example 3 | 1,081 | 1,220 | 139 | 112,693 | 433 |
| Comparative Production | 1,072 | 1,356* | 284 | 32,719 | 782 |
| Example 1 | | | | | |
| Comparative Production | 1,070 | — | — | 104,107 | 284 |
| Example 2 | | | | | |

*Crystal dispersed state

As seen from Table 1, the diimmonium salt compounds in Production Examples 1 to 3 were individually in an association form and exhibited a maximum absorption wavelength that had shifted about 20 to 150 nm to the longer wavelength side from the maximum absorption wavelength of each compound in a dissolved state. In contrast, the diimmonium salt compound in Comparative Production Example 1 was in a crystal dispersed state and exhibited a maximum absorption wavelength of 1,356 nm, that had shifted 284 nm to the longer wavelength side from the maximum absorption wavelength of the compound in a dissolved state. The amount of change in the shifting was so large that the near infrared absorption effect was markedly poor. The diimmonium salt compound in Comparative Production Example 2 was in a dissolved state even at a concentration of 100 mg/L and exhibited a maximum absorption wavelength of 1,070 nm. Even when the concentration was further increased, no shifting of the maximum absorption wavelength to the longer wavelength side occurred.

Further, as can be seen from FIG. 1, the diimmonium salt compound dispersions in Production Examples 1, 2, and 3 individually exhibit a sharp absorption band having a small half band width and have excellent near infrared absorption effect, as compared with the dispersion in Comparative Production Example 1.

### Example 1

### Preparation of Near Infrared Shielding Filter

0.5 Part of the N,N,N′,N′-tetrakis{p-di(cyclohexylmethyl)aminophenyl}-p-phenylenediimmonium hexafluorophosphate obtained in Production Example 1, 9.5 parts of toluene, and 70 parts of zirconia beads having a particle size of 0.3 mm were placed in a 50-ml glass vessel and subjected to shaking by means of a paint shaker for 2 hours, and then the zirconia beads were filtered off, and the resultant filtrate was diluted with toluene so that the concentration became 100 mg/L to obtain a diimmonium salt compound dispersion. 40 parts of the obtained diimmonium salt compound dispersion was added to a solution of 30 parts of an acrylic lacquer resin (registered trademark: THERMOLAC LP-45 M; manufactured by Soken Chemical & Engineering Co., Ltd.), 15 parts of methyl ethyl ketone, and 15 parts of toluene. The resultant solution was applied onto a commercially available general-purpose polymethacrylic resin film (thickness: 50 μm) using a bar coater having a gap size of 46 μm. Then, the applied solution was dried at a temperature of 100° C. for 3 minutes to obtain a near infrared shielding filter.

### Example 2

A near infrared shielding filter was prepared in substantially the same manner as in Example 1 except that, instead of N,N,N′,N′-tetrakis{p-di(cyclohexylmethyl)aminophenyl}-p-phenylenediimmonium hexafluorophosphate, the N,N,N′,N′-tetrakis{p-di(3-fluoropropyl)aminophenyl}-p-phenylenediimmonium hexafluorophosphate obtained in Production Example 2 was used.

### Comparative Example 1

A near infrared shielding filter was prepared in substantially the same manner as in Example 1 except that, instead of N,N,N′,N′-tetrakis{p-di(cyclohexylmethyl)aminophenyl}-p-phenylenediimmonium hexafluorophosphate, the N,N,N′,N′-tetrakis{p-di(n-propyl)aminophenyl}-p-phenylenediimmonium hexafluoroantimonate obtained in Comparative Production Example 1 was used.

### Comparative Example 2

A near infrared shielding filter was prepared in substantially the same manner as in Example 1 except that, instead of N,N,N′,N′-tetrakis{p-di(cyclohexylmethyl)aminophenyl}-p-phenylenediimmonium hexafluorophosphate, the N,N,N′,N′-tetrakis{p-di(n-propyl)aminophenyl}-p-phenylenediimmonium tetrafluoroborate obtained in Comparative Production Example 2 was used.

### Test Example 2

### Evaluation of Performance of Near Infrared Shielding Filter

With respect to each of the near infrared shielding filters obtained in Examples 1 and 2 and Comparative Examples 1 and 2, a haze (turbidity) was measured by means of haze meter NDH 5000 (manufactured by NIPPON DENSHOKU INDUSTRIES CO., LTD.). These near infrared shielding filters were subjected to heat resistance test by storing them in an atmosphere at a temperature of 80° C., and after a lapse of a predetermined period of time, the resultant filters were subjected to measurement of transmittances at respective wavelengths of 1,000 nm and 480 nm by means of a spectrophotometer. Further, these near infrared shielding filters were subjected to moist heat resistance test by storing them in an atmosphere at a temperature of 60° C. and a humidity of 95%, followed by measurement of transmittances at respective wavelengths of 1,000 nm and 480 nm in the same manner as in the heat resistance test. The results of the measurement of haze, the heat resistance test, and the moist heat resistance test are respectively shown in Table 2, Table 3, and Table 4.

TABLE 2
Results of haze measurement

| | Haze |
|---|---|
| Example 1 | 1.05 |
| Example 2 | 1.24 |
| Comparative Example 1 | 4.15 |
| Comparative Example 2 | 0.94 |

TABLE 3
Results of 80° C. heat resistance test

| | 1,000 nm Transmittance (%) | | | 480 nm Transmittance (%) | | |
|---|---|---|---|---|---|---|
| | | After | Amount | | After | Amount |
| | Initial | 500 h | of change | Initial | 500 h | of change |
| Example 1 | 2.3 | 2.5 | 0.2 | 68.1 | 68.0 | −0.1 |
| Example 2 | 3.5 | 3.9 | 0.4 | 67.3 | 67.0 | −0.3 |
| Comparative | 18.1 | 18.2 | 0.1 | 58.2 | 58.1 | −0.1 |
| Example 1 | | | | | | |
| Comparative | 2.1 | 6.5 | 4.4 | 77.1 | 69.4 | −7.7 |
| Example 2 | | | | | | |

TABLE 4
Results of 60° C., 95% moist heat resistance test

| | 1,000 nm Transmittance (%) | | | 480 nm Transmittance (%) | | |
|---|---|---|---|---|---|---|
| | | After | Amount | | After | Amount |
| | Initial | 500 h | of change | Initial | 500 h | of change |
| Example 1 | 2.3 | 2.9 | 0.6 | 68.1 | 67.6 | −0.5 |
| Example 2 | 3.5 | 3.7 | 0.2 | 67.3 | 67.1 | −0.2 |
| Comparative | 18.1 | 18.2 | 0.1 | 58.2 | 57.4 | −0.8 |
| Example 1 | | | | | | |
| Comparative | 2.1 | 7.9 | 5.8 | 77.1 | 69.1 | −8.0 |
| Example 2 | | | | | | |

As apparent from Table 2, the near infrared shielding filters in Examples 1 and 2, each containing an association form of the diimmonium salt compound, exhibited more excellent transparency than that of the filter in Comparative Example 1 containing the diimmonium salt compound in a crystal dispersed state, and exhibited transparency equivalent to that of the filter in Comparative Example 2 containing the diimmonium salt compound in a dissolved state. Further, as apparent from Tables 3 and 4, the near infrared shielding filters in Examples 1 and 2 had high near infrared absorptive power, as compared with the filter in Comparative Example 1, and had excellent heat resistance and excellent moist heat resistance, as compared with the filter in Comparative Example 2.

### INDUSTRIAL APPLICABILITY

The near infrared absorbent dye of the present invention has excellent heat resistance and excellent moisture resistance and suffers no lowering in the near infrared absorptive power over a long period of time. The near infrared shielding filter containing the near infrared absorbent dye can be used in various applications of PDP, automobile glass, construction glass, and the like, and is especially preferably used as a near infrared shielding filter for PDP.

1. A near infrared absorbent dye, comprising an association form of a diimmonium salt compound of formula (1): 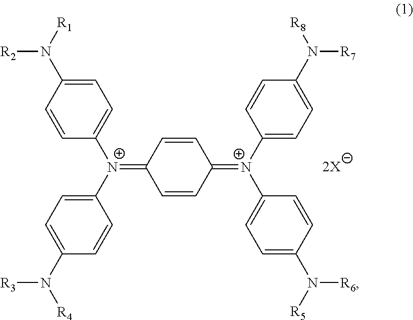
wherein
R₁ to R₈ are the same or different and each represents an organic group, and
X⁻ represents an anion.

2. The dye of claim 1, wherein X⁻ in formula (1) is a hexafluorophosphate ion.

3. The dye of claim 1, wherein at least one of R₁ to R₈ in formula (1) is a linear or branched C₁-C₁₀ alkyl group optionally substituted with a halogen atom, a C₃-C₁₂ cycloalkyl group, or a C₃-C₁₂ cycloalkyl-C₁-C₁₀ alkyl group in which the cycloalkyl ring is optionally substituted.

4. The dye of claim 3, wherein at least one of R₁ to R₈ in formula (1) is a cycloalkyl-alkyl group represented by formula (2): 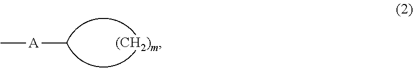
wherein
A represents a linear or branched alkyl group having 1 to 10 carbon atoms, and
m represents an integer of 3 to 12.

5. The dye of claim 4, wherein the cycloalkyl-alkyl group of formula (2) is a cyclohexylmethyl group.

6. The dye of claim 3, wherein at least one of R₁ to R₈ in formula (1) is a monohalogenoalkyl group of formula (3):
—CₙH₂ₙ—CH₂Y (3)
wherein
n represents an integer of 1 to 9, and
Y represents a halogen atom.

7. The dye of claim 6, wherein the monohalogenoalkyl group of formula (3) is a 3-fluoropropyl group.

8. The dye of claim 3, wherein at least one of R₁ to R₈ in formula (1) is an iso-butyl group.

9. A near infrared shielding filter, comprising the dye of claim 1.

10. A near infrared absorbing composition comprising a diimmonium salt in an association state dispersed in an organic solvent,
wherein the diimmonium salt is of formula (1): 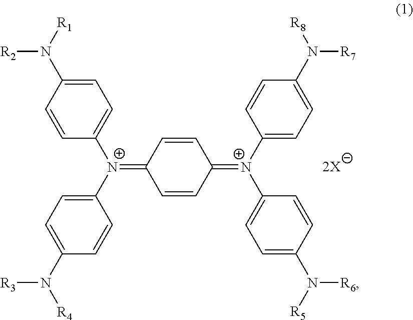
wherein
R₁ to R₈ are the same or different and each represents an organic group, and
X⁻ represents an anion.

11. The composition of claim 10, wherein X⁻ in formula (1) is a hexafluorophosphate ion.

12. The composition of claim 10, wherein at least one of R₁ to R₈ in formula (1) is a linear or branched C₁-C₁₀ alkyl group optionally substituted with a halogen atom, a C₃-C₁₂ cycloalkyl group, or a C₃-C₁₂ cycloalkyl-C₁-C₁₀ alkyl group in which the cycloalkyl ring is optionally substituted.

13. The composition of claim 12, wherein at least one of R₁ to R₈ in formula (1) is a cycloalkyl-alkyl group of formula (2): 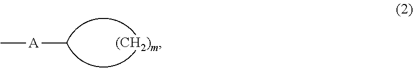
wherein
A represents a linear or branched alkyl group having 1 to 10 carbon atoms, and
m represents an integer of 3 to 12.

14. The composition of claim 13, wherein the cycloalkyl-alkyl group of formula (2) is a cyclohexylmethyl group.

15. The composition of claim 12, wherein at least one of R₁ to R₈ in formula (1) is a monohalogenoalkyl group of formula (3):
—CₙH₂ₙ—CH₂Y (3)
wherein
n represents an integer of 1 to 9, and
Y represents a halogen atom.

16. The composition of claim 15, wherein the monohalogenoalkyl group of formula (3) is a 3-fluoropropyl group.

17. The composition of claim 12, wherein at least one of R₁ to R₈ in formula (1) is an iso-butyl group.

18. A diimmonium salt compound of formula (4): 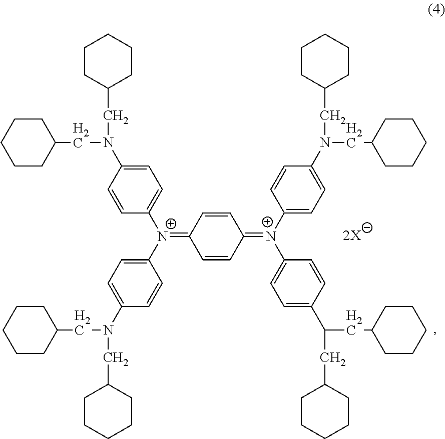
wherein X⁻ represents an anion.

19. The compound of claim 18, wherein X⁻ in formula (4) is a hexafluorophosphate ion.

20. A diimmonium salt compound of formula (5): 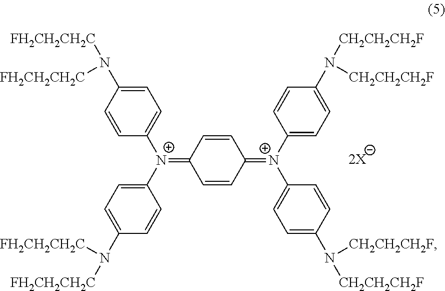
wherein X⁻ represents an anion.

21. The compound of claim 20, wherein X⁻ in formula (5) is a hexafluorophosphate ion.

22. The dye of claim 2, wherein at least one of R₁ to R₈ in formula (1) is a linear or branched C₁-C₁₀ alkyl group optionally substituted with a halogen atom, a C₃-C₁₂ cycloalkyl group, or a C₃-C₁₂ cycloalkyl-C₁-C₁₀ alkyl group in which the cycloalkyl ring is optionally substituted.

23. The dye of claim 22, wherein at least one of R₁ to R₈ in formula (1) is a cycloalkyl-alkyl group represented by formula (2): 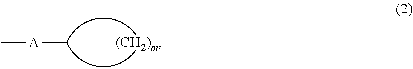
wherein
A represents a linear or branched alkyl group having 1 to 10 carbon atoms, and
m represents an integer of 3 to 12.

24. The dye of claim 23, wherein the cycloalkyl-alkyl group of formula (2) is a cyclohexylmethyl group.

25. The dye of claim 22, wherein at least one of R₁ to R₈ in formula (1) is a monohalogenoalkyl group of formula (3):
—CₙH₂ₙ—CH₂Y (3)
wherein
n represents an integer of 1 to 9, and
Y represents a halogen atom.

26. The dye of claim 25, wherein the monohalogenoalkyl group of formula (3) is a 3-fluoropropyl group.

27. The dye of claim 22, wherein at least one of R₁ to R₈ in formula (1) is an iso-butyl group.

28. The composition of claim 11, wherein at least one of R₁ to R₈ in formula (1) is a linear or branched C₁-C₁₀ alkyl group optionally substituted with a halogen atom, a C₃-C₁₂ cycloalkyl group, or a C₃-C₁₂ cycloalkyl-C₁-C₁₀ alkyl group in which the cycloalkyl ring is optionally substituted.

29. The composition of claim 28, wherein at least one of R₁ to R₈ in formula (1) is a cycloalkyl-alkyl group of formula (2): 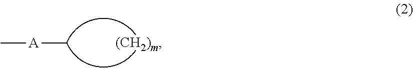
wherein
A represents a linear or branched alkyl group having 1 to 10 carbon atoms, and
m represents an integer of 3 to 12.

30. The composition of claim 29, wherein the cycloalkyl-alkyl group of formula (2) is a cyclohexylmethyl group.

31. The composition of claim 28, wherein at least one of R₁ to R₈ in formula (1) is a monohalogenoalkyl group of formula (3):
—CₙH₂ₙ—CH₂Y (3)
wherein
n represents an integer of 1 to 9, and
Y represents a halogen atom.

32. The composition of claim 31, wherein the monohalogenoalkyl group of formula (3) is a 3-fluoropropyl group.

33. The composition of claim 28, wherein at least one of R₁ to R₈ in formula (1) is an iso-butyl group.
